(19) United States
(12) Patent Application Publication
(10) Pub. No.: US 2023/0360755 A1
(43) Pub. Date: Nov. 9, 2023

(54) COMPUTERIZED METHOD FOR DETERMINING NUTRITIONAL SUPPLEMENT DELIVERY

(71) Applicant: BETTER & BETTER CO., New York, NY (US)

(72) Inventor: Vladimir Vukicevic, Long Island City, NY (US)

(21) Appl. No.: 17/739,277

(22) Filed: May 9, 2022

(51) Int. Cl. G16H 20/00; G16H 50/30; G16H 10/60; G16H 10/20

(52) U.S. Cl. CPC G16H 20/00; G16H 50/30; G16H 10/60; G16H 10/20

(57) ABSTRACT

The present computerized method provides for electronically tracking user health markers and providing user supplement product(s) with consumption based on pre-existing user habits. The computerized method includes receiving user data including first user health data and analyzing the data relative to a plurality of health marker data sets. Therein, the method includes determining, based on the analysis, at least one health deficient marker associated with the user health data indicating a health deficiency and based thereon selecting a first supplement for addressing the health deficient marker. The computerized method determines a user pre-existing habit and determines a first consumer product infused with the first supplement consistent the pre-existing habit of the user. After a consumption duration, the computerized method receives updated user data including second user health data and analyzes the data, computing a variance value of the health deficiency after the consumption duration.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### FIELD OF INVENTION

The disclosed technology relates generally to health tracking software and more specifically to computerized testing and evaluation of user health data and nutritional supplement consumption relative to determined user habits.

### BACKGROUND

It is not uncommon for individuals to be nutritionally-deficient. These deficiencies arise from any number of reasons, including diet, environment, seasons, genetics, to name a few. It is well established that users can take nutritional supplements to correct these deficiencies.

Problems continually arise in user compliance. We see this problem not only with prescription medicines, but also nutritional supplements. One problem is that taking pills or supplements adds a new action or habit for the user. Where a user is not accustomed to taking pills, it is common to forget to take the pills.

Users are able to quickly and efficiently determine their health data using any number of third-party services. Examples can include visiting a doctor and having a blood test performed. Lab analysis can determine any potential deficiencies for the user, but then it becomes incumbent upon the user to either coordinate with a health care provider or upon their own volition, to find the supplement and begin taking the supplement, undertaking a new habit.

It is also becoming known to infuse consumer products with supplements. For example, U.S. Pat. No. 3,992,519 describes infusing toothpaste with a vitamin component, such as vitamin E. In another example, U.S. Pat. No. 6,207,137 describes a dental formation, e.g. toothpaste, having a vitamin C active component.

There is no current solution in the marketplace that merges the disparate supplement delivery techniques available. Additionally, there are no current solutions that direct consumers for health improvements based on existing user habits. The current supplement consumption regime relies on taking pills, requiring the new behavior that contributes to non-compliance.

Additionally, there are no current technical solutions for tracking effectiveness and long-term effects of supplement consumption. Current techniques would be the iterative process of taking a supplement for a period of time, then visiting the doctor again for a second blood test. Analysis of the second blood test can then determine if the underlying deficiency was corrected.

Where there are infused consumer products, there are no software solutions for analyzing health data and tying consumption to pre-existing habits. Additionally, there lacks any software solution for monitoring or iterative updating of health information.

### BRIEF DESCRIPTION

The present method is a computerized method for improving or correcting user health deficiencies by analyzing user data and executing operations therefrom.

The computerized method improves user health by analyzing data sets and determining consumer products that deliver nutritional supplements via consumption techniques consistent with pre-existing user habits. The computerized method operates by detecting and selecting processing operations and referencing a large data set of consumer products having nutritional supplements therein or capable of being infused, where consumption of these consumer products is within the pre-existing user habits. Therein, the computerized method and system improves user health based on existing user habits.

The computerized method operates in a computer processing environment, including server or cloud-based processing operations integrated and in communication with a product shipping/delivery system.

The computerized method receives user data relating to a specific user, the user data including user health data and activity data. For example, the user data can be blood draw data acquired from a medical lab or health care provider. In another example, the user data can be from a home or personal-use data collection or testing kit or tracking device or software. In another example, the user data can be user-submitted data, such as a survey data. In another example, the user data can more generalized data relating to the user, including for example gender, demographic, age, residency location, profession, etc.

User activity data can be any data relating to user activities. For example, user activity data can be user-entered information, including survey response data, demographic data, genealogy/genetic data, environmental data, etc. For example, user activity data can be data acquired from third party services and/or devices, such as fitness trackers, fitness apps, body value measurement devices, etc.

The computerized method analyzes the user health data in relation to health marker data sets. The health marker data sets can be reference data sets indicating appropriate or recommendation ranges for various health markers. The health markers can be individual health values, for example a specific blood value level or vitamin level reading. The health markers can also be general health values, for example blood pressure, A1C value, cholesterol number, by way of example.

The computerized method analyzes the user activity data and determines a plurality habits of the user. The habits can be any activity the user performs on a regular or routine basis, such as done one or more times daily, weekly, or any other regular interval. This pre-existing habit determination can be via the user data, via a consumer survey, or based on general knowledge. For example, user habits can include, but are not expressly limited to, tooth brushing, tooth flossing, rinsing with mouthwash, chewing gum, applying hand lotion, applying face lotion, daily shampoo application, daily conditioner application, daily make-up application, among others.

Based on the analysis, the computerized method therein determines one or more health deficiencies associated with the user health data. For example, health deficiencies can include the user being deficient in various minerals, e.g. iron, magnesium, selenium, etc. For example, health deficiencies can include the user being deficient in one or more various vitamin levels, e.g. vitamin A, vitamin B, vitamin C, vitamin E, etc.

The computerized method can therein electronically determine a supplement for addressing the health deficiency. This electronic determination and selection can use a look-up table or any other suitable reference table, referencing the supplement that addresses the health deficiency.

The computerized method further determines, using computer processing operations, consumption techniques associated with the supplement. The efficacy of some supplements can be enhanced or diminished by delivery techniques. For example, vitamin E is a fat-soluble vitamin and may need some fat or other lipid for effective absorption. For example, some minerals may be readily absorbable via topical or skin-based absorption as we well as mouth-based contact absorption. Therefore, each associated supplement includes one or more consumption techniques relating to effective distribution and absorption of the supplement by a user.

The computerized method electronically determines which consumer products are infusible with the supplement, where consumption of the consumer product and the consumption technique are consistent with the at least one habit of the user.

The computerized method additionally calculates a dosing value for the supplement to address the health deficiency. This dosing value can be based on the user health data. For example, the user health data may indicate a height and weight for the user and the dosing level can be adjusted based thereon.

The computerized method utilizes multiple consumer products for supplement delivery. Thus, the computerized method electronically determines, for each of the consumer products, a supplementation level for consumption of the consumer product. Wherein, the supplementation levels for the plurality of consumer products collectively meet the dosing value for the supplement, accounting for user consumption in accordance with existing habits.

Moreover, further electronically computing operations can account for the consumer product consumption, the supplement, and biological factors for supplement absorption. The consumer products offer varying supplement delivery techniques and each technique includes different absorption levels. Therefore, when determining the supplementation level, this calculation additionally accounts for absorption efficacies of the supplement infused therein.

The computerized method integrates with and/or communicates with a consumer product delivery system. Based on selection of the consumer product, the computerized method therein communicates with the delivery system for delivering the consumer product to the user. This delivery can be effectuated using the user data, for example if the user data includes address or delivery information.

The computerized method may operate in conjunction with one or more product infusion systems or devices. The method may include transmitting the supplantation level to the infusion system. Wherein the system infuses the consumer product consistent with the supplementation level.

In addition to the product selection and delivery, the computerized method can be an iterative system for tracking user health markers. The computerized system defines a consumption period associated with the consumer product. This consumption period can be the time period for consuming the consumer product with the supplemental infusion. The consumption period could also be a defined time period for detecting if the user's consumption of the consumer product is having a beneficial effect.

By way of example, if the consumer product is a toothpaste bottle infused with Vitamin A, the consumption duration may be 30 days based on the volume of toothpaste in the bottle, presuming twice-daily tooth brushing.

After the consumption duration, the computerized method can iterate the consumer product supplementation techniques described herein. For example, the user can provide second user health data after the consumption duration. This second user health data can be similar to the user health data.

Therein, in this embodiment, the computerized method can then analyze the second user data, determining the effectiveness of the consumer product infused with the first product. This analyze can include computing a variance value representing a change in the health deficiency of the user after at least a time period defined by the consumption duration.

Therefore, further products or variations of the same products can be recommended to the user. By way of example, if the variance value indicates a positive correction of the underlying deficiency, but it remains deficient, a new product with increased infusion levels can be recommended. In another example, if the variance value indicates a positive correction, the user can be recommended to pursue further consumption of the consumer product for a second consumption duration. In another example, if the deficiency is corrected, the user data analysis could ascertain a new deficiency and change the recommendation for a consumer product (same or different) infused with a different supplement.

In another embodiment, the computerized method may provide additive supplementation for consumer products based on user data and user activities, not specifically directed to nutritional-deficiencies. Instead of health data, computational analysis of user activity data can provide for estimating supplementation. For example, use activity data can indicate the person is highly active and training for a sporting event. The supplementation may include non-prescription supplements, CBD oil, fish oil, or other supplementation not directly related to health data. In another example, the user may request dosing or micro-dosing of a prescription or a controlled substance, having proper medical clearance. This supplementation can be based on activities, for example the person suffering from an anxiety disorder and not specifically originating from a biological specimen, for example a blood draw.

### BRIEF DESCRIPTION OF THE DRAWINGS

FIG. 1 illustrates one embodiment of a system providing for electronically detecting and tracking user health markers.

FIG. 2 illustrates one embodiment of a computing system providing for detecting user health markers.

FIG. 3 illustrates one embodiment of a computing system providing for tracking and updating user health markers.

FIGS. 4A and 4B illustrate a data flow diagram for the computerized method of electronically tracking user health markers.

A better understanding of the disclosed technology will be obtained from the following detailed description of the preferred embodiments taken in conjunction with the drawings and the attached claims.

### DETAILED DESCRIPTION

The method and system herein provides tracking user health markers and selecting and distributing supplement-infused consumer product(s) for supplement-dosing and product consumption based on pre-existing user habits.

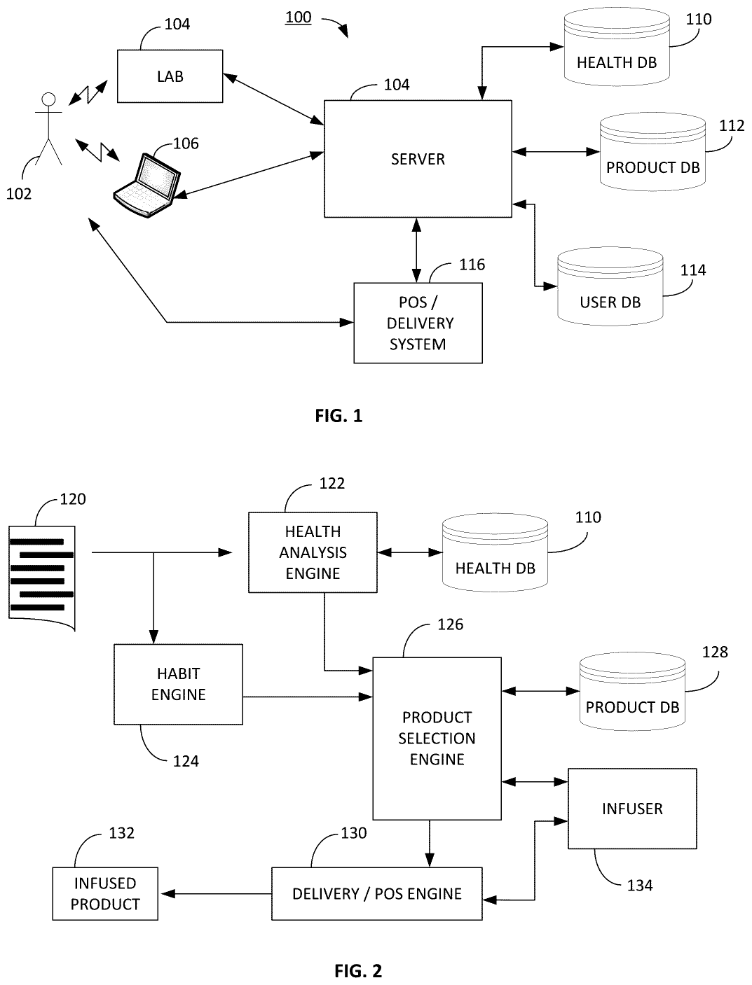

FIG. 1 illustrates a system 100 including a server 102. The server 102 communicates with a lab 104 and at least one computing device 106 via one or more networked connections. A user 108 submits personal information via the lab 104 and/or the computing device 106.

The server 102 also communicates with various databases, including health database 110, product database 112, and user database 112. The server additionally communicates with a point-of-sale (POS)/delivery system computing system 116.

The server 102 can be one or more process devices, typically network-based processing devices. The server 102 performs processing operations as described herein in response to the executable instructions, performing processing operations and calculations based on various data sets provided or accesses via networked connections.

For example, in one embodiment, the server 102 can be a cloud-based operation with processing operations in a distributed computing environment. For example, in one embodiment, the server 102 can be a dedicated computer or processing device with direct access via network communication protocols. Where the server 102 can use existing or known processing hardware and processing elements, the computerized processing algorithm provides a unique functional result as noted herein.

The databases 110, 112, and 114 can be local or network-accessible database(s) having data stored therein. The databases 110, 112, and 114 can operate consistent with known data storage and retrieval operations. The databases 110, 112, and 114 can also be one or more data storage locations either centrally located or disposed across a distributed network.

In further embodiments, the health database 110 and the user database 114 may also include or require security or authentication to protect patient records, including security for conforming with any and all health information privacy protection requirements.

The lab 104 can be a commercial laboratory, such as a third-party lab, providing patient health data to the server 102. For example, the lab 104 may receive a biological specimen from the user. In one embodiment, the biological specimen could be a blood sample. The lab 104 can therein perform processing analysis of the biological specimen to extract the biological information.

The lab 104 can also be a more general term for any device or devices capturing user health information and electronically connected for sharing the health information. For example, the lab 104 can be a smart phone or smart watch collecting user movement and health data, for example tracking sleep data and recording sleep patterns. In another example, the lab 104 can be a home health tracking device such as a glucose monitoring application by way of example.

The computer 106 can be any suitable local processing device, such as a laptop or desktop computer, a smart phone, by way of example. The computer 106 operates using known communication techniques for facilitating the user 108 to interact with the server 102. For example, one embodiment may include a web browser running on the computer 106, the browser displaying a questionnaire or survey for the user 108. As the user 108 enters answers, the data is transmitted to the server 102 across the communication network, e.g. Internet. In another example, the server 102 can facilitate data exchange between the POS/Delivery system 116 and the user 108 via the computer 106.

The POS/Delivery system 116 may be a stand-alone product delivery system or can be tied into a third-party system or network. For example, in one embodiment, the product delivery system 116 can be integrated into the Amazon® webservices system facilitating product ordering and delivery.

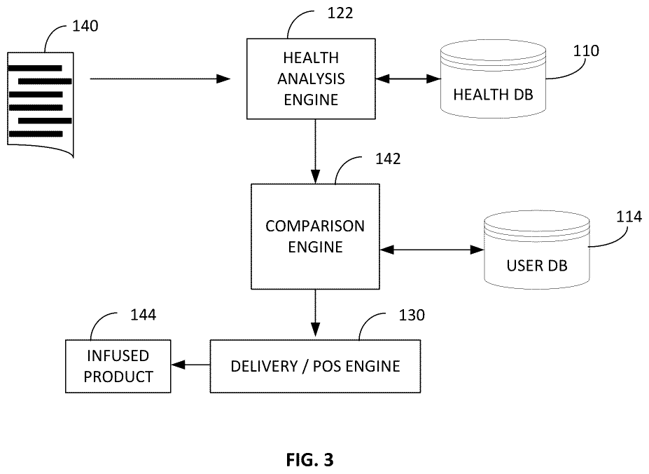

FIG. 2 illustrates the processing elements and operations for the computerized method for electronically tracking user health markers. The computerized method not only tracks user health markers, but also facilitates improving designated health deficiency markers based on product recommendations consistent with pre-existing user habits.

In the computerized method, user health data 120 is electronically transmitted to a health analysis engine 122. The health analysis engine 122 includes executable instructions to analyze the user health data 120 and determine at least one health deficiency of the user. The health analysis engine 122 references the health database 110 having health marker data sets stored therein.

The health data 120 can include, for example, biological information from a biological specimen of the user, such as from a laboratory or third-party analysis engine. The health data 120 can also include user survey response data, user demographic data, or any other suitable data relating to either the user or the user's environment.

For example, the survey data can include questions about the user's lifestyle and/or environment, as well as background. In one example, it can be useful to understand where the person lives, how often they go outside, and their-skin type. In this example, if the person is deficient in Vitamin D, it can be useful to know if the person lives in a cold climate with limited sun exposure or chances for being outside versus living in a warm climate. For example, person A living in Bangor Maine in the winter will have a completely different Vitamin D profile from person living in Miami Florida.

The health marker data sets are data sets indicating ranges or values associated with health markers. By way of example, a health marker data can be a specific measurement or range of values of a vitamin or mineral in the user's blood. For example, the health marker data can indicate a preferred range of vitamin C levels for a specific user.

The health marker data sets relate to a large variety of health markers, including for example but not expressly limited to, vitamin levels, mineral levels, lipid levels, blood value levels, etc.

The health analysis engine 122 compares the health data 120 relative to the health marker data sets to determine one or more health deficiencies of the user. Using the above example of Vitamin C, if the user data 120 indicates a Vitamin C value below the health marker data set range, the engine 122 determines the health deficiency indicates a vitamin C deficiency. It is additionally recognized the health deficiency and the associated supplement may not be a direct one-to-one correlation, for example in some embodiments having one health deficiency can be addressed by a different supplement that can assist in the user's ability to cure the health deficiency.

The health analysis engine 122 can then further determine a supplement to address the health deficiency. In the above example of Vitamin C deficiency, the supplement determination can be increased consumption of Vitamin C.

The health analysis engine 122 may further determine one or more health deficiency based on the lifestyle data, such as may be acquired via the user data. Using the above example of Vitamin D and sun exposure, the engine 122 can determine a Vitamin D deficiency based on the lifestyle data of living in a cold-weather climate.

Additionally, each nutritional supplement includes one or more consumption techniques. The consumption techniques are known manners in which the supplement may be received by a user, via dosing or micro-dosing exposures. For example, consumption techniques can include but are not expressly limited to topical, buccal, sublingual, inhalation, among others.

As part of the computerized method noted herein, the user data 120 is also distributed to a habit engine 124. The habit engine 124 can be one or more processing engines performing data analysis in response to executable instructions.

The habit engine 124 analyzes the user activity of the user data to determine or validate at least one pre-existing user habit. The user data 120 can be medical or health data, but can also be user survey or user input data such as via the computer 106 of FIG. 1. In the example of survey data, the user can be presented with various survey questions about daily or routine activities. In another example, the user data 120 can include user profile data from one or more third-party systems indicating user patterns or behaviors. Examples can include tracking applications or software, such as found with a smart phone or smart watch. Other examples can include personal monitoring devices, for example a glucose monitoring device or other similar type device.

The prior art supplement industry relies on the user undertaking a new habit, such as taking a daily pill. The habit engine 124 improves tracking user health markers by tying in nutritional supplement consumption with existing user behaviors or habits. Therefore, the present computerized method includes recognizing and/or determining the user behaviors or habits as conducted by the habit engine 124.

In one example, if the user activity data of the user data 120 is survey data, the habit engine 124 can review the survey data to ascertain the habits. In one embodiment, the survey can be tailored to acquiring habit data, including specific questions if the user engages in various activities.

Habits can be generally known as any action or sequence of actions the user undertakes on a daily or regular/standard basis. Habits can include brushing one's teeth, flossing, applying hand or face lotion, shaving, by way of example. The above examples are not an exclusive or exhaustive list of habits, but representative in nature only.

Based on analyzing the user data 120, the habit engine 124 therein determines at least one pre-existing habit of the user. In one example, the pre-existing habit determination can be that the user has the habits of teeth brushing, flossing, and daily shaving.

The habit engine 124 therein provides the one or more pre-existing habits to a product selection engine 126. The product selection engine 126 additionally receives the first supplement from the health analysis engine 122.

The product selection engine 126 is a computational engine based on executable instructions that selects one or more consumer product for the user based on the pre-existing habit of the user, the supplement determination, and the associated consumption technique(s). In one embodiment, a product database 128 includes a list or reference of various consumer products either having a supplement added therein or capable of being mixed with one or more supplements.

In one example, the product database 128 can be a proprietary database of products offered by one or more vendors. In another embodiment, the product database can be a general commercial database of consumer products.

The production selection engine 126 includes processing routines for comparing the supplement selected by the health analysis engine 122 and the habit(s) from the habit engine 124. The product selection engine 126 therein determines one or more consumer products capable of being infused with or already having the supplemental infused therein.

The product selection engine 126 communicates with a delivery/POS engine 130. The engine 130 may be within the POS/Delivery system 116 of FIG. 1. The delivery engine 130 may additionally receive the user data 120 or at least a portion of the user data 120 such as billing and/or shipping information.

Having selected at least one consumer product with at least one supplement therein, the delivery/POS engine 130 can therein deliver the infused product 132 to the consumer. The engine 130 can include generating shipping instructions, shipping labels, or communicating with a product shipping company for shipping and handling operations to physically deliver the infused product to the user.

In one embodiment, the computerized method may interact with the user prior to product shipment. For example, one embodiment may include transmitting a product recommendation to the user 108 via the computing device 106 of FIG. 1. The product recommendation may include a dedicate hyperlink or other electronic communication technique to generate an order request for electronic purchase of suggested consumer product. Therefore, in one embodiment the product selection engine 126 and/or the delivery/pos engine 130 may interact with the user prior order and delivery of the supplement-infused consumer product.

In one embodiment, the system of FIG. 2 may additionally include an infusion device 134. The infusion device 134 is one or more physical machines or devices for infusion supplement into a product. For example, if the supplement is to be added to toothpaste, the infuser can inject or mix the toothpaste with a liquid or gel of the supplement. Moreover, the infusion device 134 can regulate or modify the amount of supplement infused into a product based on a supplementation level, for example adding a small, medium, or large dosage of the supplement in response to infusion instructions.

The computerized method operates in an iterative manner for tracking user health markers. FIG. 2 is the initial phase, selecting and distributing infused product(s) 132 to the consumer, where consumption of the product(s) conforms to the user's pre-existing habits.

Associated with either the consumer product or the nutritional supplement is a consumption duration. The consumption duration can the estimated time to consume the product. For example, if the product is a tube of supplement-infused toothpaste, the duration may be 30 days based on estimating twice-daily teeth brushing. In another example, if the product a bottle of supplement-infused hand lotion, the duration may be 20 days based on estimating thrice-daily hand moisturizing.

The consumption duration can also be associated with a time period for exposure or user dosing of the supplement-infused product. For example, using the example of toothpaste, the bottle may have enough paste to last forty-five days, but the consumption duration is thirty day based on the estimated dosing response and active-ingredient level of the supplement.

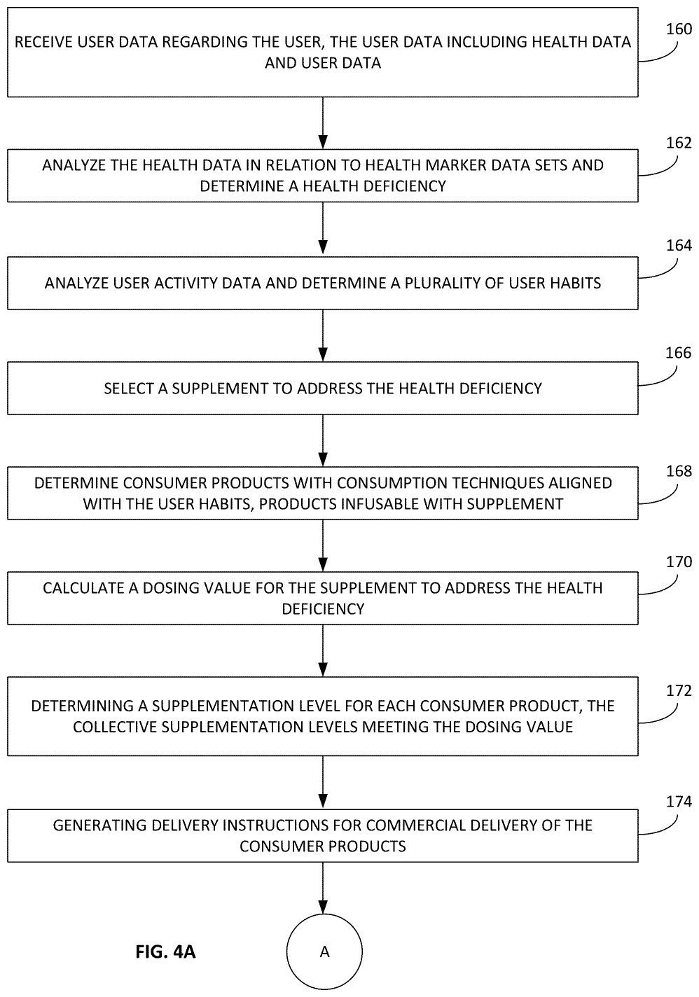

The present computerized method not only distributes an infused consumer product aligned with one of the user's pre-existing habits, the computerized method operates to track user health markers upon completion of the consumption duration. Therefore, FIG. 3 illustrates the computerized method after the consumption duration.

The computerized method receives second user data 140, being updated user data. The updated user data 140 may include similar identifier data as the user data 130 of FIG. 2, but includes updated health data after the user has continued exposure to the nutritional supplement(s). Similar to user data 120 of FIG. 2, the updated user data 140 may be received from any number of sources, including laboratories results, user interaction with at-home or clinical measurement devices, electronic health tracking apps or devices, interactive survey questions, or other input techniques as recognized by one skilled in the art.

The updated user data 140 is received in the health analysis engine 122. Similar to computing techniques noted above regarding FIG. 2, the health analysis engine 122 therein analyzes the updated user data. This data analysis can include determining the various measurement values or ranges for the user.

The health analysis engine 122 can supplement the computation using health marker data sets from the health database 110. The analysis of the updated user data is then provided to a comparison engine 142. The comparison engine 142 may be a set of executable instructions performed within the server 102 of FIG. 1.

The comparison engine 142 therein analyzes the second health user data relative to the first user health data acquired prior to the consumption period. In one embodiment, the first user health data is stored in the user database 114 and accessed by the comparison engine 142.

In the analysis, the comparison engine 142 determines a variance value of the health deficiency of the user after supplement dosing via the consumer product over the consumption duration. The variance value can be any suitable type of data indicator indicating a change in the underlying value or values that facilitated the prior supplement assertation.

In one embodiment, the variance value can be a numeric difference in a specific nutrient or other measurement between the first user health data and the second user health data. In another embodiment, the variance value can be a percentage change in value between health data sets. In another embodiment, the variance value can be a more general positive or negative direction indicator, indicating the general direction of value adjustment between data sets.

In one embodiment, the computing and processing operations occurring after the consumption duration can also address or seek additional information on the user's habit. For example, one embodiment can include a survey or other type of inquiry with the user via a graphical user interface or web browser. The survey can include questions indicating if the user maintained the habit during the consumption period. For example, if the infused product was an infused hand lotion and the user failed to maintain the habit of applying hand lotion, the computerized system may modify the functional logic for addressing the effectiveness of the first consumer product away from the product, but instead on the effectiveness of the selected habit, as well as determine a different habit for a second consumer product.

In FIG. 3, the tracking of user health data iterates the supplemental process. The comparison engine 142 can either by itself or in conjunction with a product selection engine (126 of FIG. 2) select a second infused product for the user.

The second selected product may be the same as the first product, for example seeking the user to undertake further supplement dosing via the same delivery technique. The second selected product may also be the same product with a variance of the supplement amount in the product.

In another embodiment, the comparison engine 142 may determine the user is no longer deficient in a specific vitamin, mineral, or other health marker. Whereby further analysis can determine any other or newly presenting health deficient marker. If a new health deficiency is discovered, the second product could be a completely different product or same product as the first product with a supplement now directed towards the new health deficient marker.

Similar to the FIG. 2 system, when the comparison engine 142 determines the product and/or infused product with supplement therein, the infused product 144 is then made available to the user via the delivery/pos engine 130.

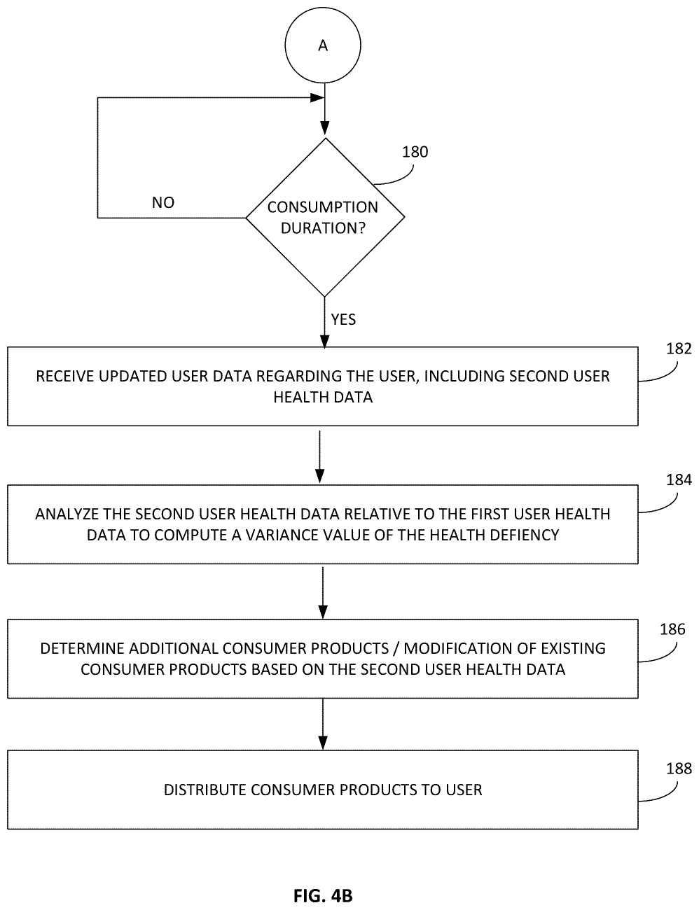

Therefore, the computerized method is an iterative process for electronically tracking user health markers based on supplement distribution confirming to existing user consumption habits.

Where FIGS. 1-3 illustrate various processing environments operative to electronically track user health markers, FIG. 4A is a first phase of the computerized method. The methodology steps may be performed, in one embodiment, using the systems of FIGS. 1-3 above.

The FIG. 4A methodology provides for electronically determining nutritional supplement delivery via consumer products. Step 160 is receiving user data regarding the user, the user data including health data and user activity data. As noted above, the user data can be received from any suitable source, including for example laboratory testing data, user-entered data, smart devices or smart testing devices, etc.

Step 162 is to analyze the health data in relation to health marker data sets and determine a health deficiency. The health marker data sets indicate standard values or ranges of values for different health markers for individuals. The health marker data sets can also be based on additional health or demographic information, for example health values can differ based on the gender of the user, as well as the age of the user. In another example, demographic information can also affect the values and/or ranges.

In one embodiment, a health deficiency can be determined or ascertained even if health data is within a specific range. For example, demographic and geographic data can indicate a likelihood of a potential deficiency. In another example, the user may not be technically deficient, but the health data may indicate a lower-end level or a falling level for a particular measurement and therein estimate or select a supplement not based on a deficiency determined solely by number-based benchmarks, but rather a holistic analysis of the user data.

Step 164 is analyzing the user activity data and determining user habits. As noted above, the user activity data can be acquired from any number of suitable sources, including tracking applications and devices, surveys or questionnaire, shopping or other user histories, etc.

Step 166 is selecting a supplement to address the health deficiency. By way of example, if the deficiency is a low magnesium level, the first supplement can be a magnesium supplement. Step 166 may be a comparative step by denoting a health data reading or value outside of a specific range. Step 166 may also be a processing routine estimating a predictive supplement for improving the user's health.

Step 168 is determining consumer product with consumption techniques aligned with the user's habits. The consumer products are also capable of being infused with the supplement. By way of example, if the user's habits are noted as twice-daily toothbrushing, daily shampoo usage, facial shaving, and daily hand lotion usage, step 168 can reference these habits and determine the appropriate consumer products, such as toothpaste, shampoo, shaving cream, and hand lotion.

In addition to the selection of products, the computerized method further accounts for the proper supplement intake via the various forms. Therefore, step 170 is calculating a dosing value for the supplement. The dosing value represents a calculation or value for supplement intake relative to the user. This dosing value can be based on recommended health guidelines. This dosing value can also be based on user data, such as adjusted for the user's body weight and height, gender, and other factors.

Where the user receives the supplement intake via consumer products, the dosing value must therefore be allocated to the individual consumer products. Step 172 is determining a supplementation level for each consumer product, the collective supplementation levels meeting the dosing value.

As noted above, the consumer products are all different products and therefore have different ingestion and dosing rates. By way of example, supplement intake via toothpaste may generate higher dosing through buccal exposure than shampoo and transdermal exposure. The supplementation levels relate to each individual consumer product, accounting for the supplement dosing levels per habit activity and the number of per-day habit activities. The method therefore generates the dosing value for the user and this dosing value is distributed to the various consumer products so the collective exposure to the consumer products generates the periodic, e.g. daily, dosing of the supplement.

Step 174 is then to communicate with a consumer product delivery system and transmit delivery instructions to deliver the first consumer product to the user.

In the method of FIG. 4A, the user therefore receives supplements not through undertaking a new behavior, but rather delivered in due course to the user concurrent with the user's existing habits of consuming consumer products.

In one embodiment, the method of FIG. 4A may additionally communicate and operate with the infuser 134 of FIG. 3. In this embodiment, the method may include generate infusion instructions for the infuser to inject or mix the applicable supplement with the consumer product. The infusion instructions can include a supplementation level, being a concentration level for mixing with the consumer product.

In one example, the supplement may be Vitamin D, the consumer product may be toothpaste, and the supplementation level for the toothpaste may be 2500 IUs of Vitamin D. In this example, the dosing level may be 5,000 IUs of Vitamin D and the habit-based selection of consumer products includes toothpaste, mouthwash and shampoo. In this example, the 5,000 IU dosing level is divided into 2500 IUs for the toothpaste, 1500 IUs for the mouthwash, and 1000 IUs for the shampoo.

The infuser 134 of FIG. 2 can receive instructions to mix the toothpaste with a supplement mixture generating a daily supplement intake of the 2500 IUs per day. The user is recognized as having the habit of twice-daily tooth brushing and the standards toothpaste usage is 0.25 grams of toothpaste per brushing. Therefore, the supplementation level mixes the toothpaste with the supplement liquid or gel to generate the 1250 IU dosing per 0.25 grams of toothpaste.

The infuser 134 of FIG. 2 may additionally operate with other consumer products. Maintaining the same example, the infuser may then mix the supplement with mouthwash to generate the 1500 IU daily exposure and then mix the supplement with shampoo to generate the 1000 IU daily exposure.

The supplementation level is calculated based on the consumer product and anticipated exposure. Therefore, the supplement amounts infused to each of the products can vary based on the properties of the medium of the consumer product.

In one embodiment, the intake of the various consumer products can also be attuned based on a consumption protocol. The consumption protocol provides instructions for the user based on dosing values for the supplement and the various consumer products. For example, the consumption protocol can include instructions for the amount of consumer product to use, e.g. volume of mouthwash by way of example, and the duration, e.g. gargle for 30 seconds.

Here, in FIG. 4A, the method pauses at block A for a defined time period, the consumption duration. As noted above, the consumption duration can be the estimated time for the user to consume the product, or in another embodiment can be a dosing interval defining a period of time during which the user is receiving the supplement.

In FIG. 4B, the method continues for step 180, determining if the consumption duration has expired. Upon expiration, step 182 is receiving updated user data regarding the user, including second user health data. Similar to the prior user data, this can be collected and transmitted to the server or other processing system.

In one embodiment, the computerized method can include a direct communication request to the user at or near the expiration of the consumption duration. For example, the user can be notified to schedule a second data collection appointment, e.g. lab appointment, or schedule to deliver health data upon completion of the duration. The computerized method may be a passive system receiving the second health user data or the computerized method can actively solicit and extract the second user health data at the appropriate time.

Step 184 is analyzing the second user health data relative to the first user health data to compute a variance value of the health deficiency. Step 186 is determining a next consumer product with infused supplement based on the analysis of the second user health data. For example, if the variance value indicates the user is responding to the supplement but the second user health data indicates the user remains deficient, the next consumer product can be a repeat of the first consumer product, or the same product with a different supplement dosage.

Step 188 is then distributing the next consumer product to the user. This can be via the delivery/POS engine 130 of FIG. 1.

The present computerized method may include additional routines for supplementing or improving the user health based on pre-existing habits. For example, the selection of the consumer product may include selecting supplementary consumer products that assist or compliment consumption. For example, one consumer product may be a toothpaste infused with one or more supplements. The supplementary consumer product may be a product that compliments oral hygiene, such as a toothbrush and/or dental floss. The supplementary consumer product could also include supplement infusion, allowing for further supplement dosing. In the example of dental floss, the floss could be infused with a supplement, creating addition dosing or micro-dosing exposures during use.

Herein the method and operating system executing the method builds upon the pre-existing habits of the user. The supplement does not need to be a single supplement, rather the method herein can provide for multiple supplements, whether both combined in a consumer product, or distributed across multiple consumer products.

By way of example, the computerized method can analyze the user data and recommend vitamin D toothpaste, vitamin D shaving cream, vitamin E shampoo, and zinc mouthwash.

The method provides for infusing products personalized to the specific user. The infusion is personalized both by the supplement or supplement types, as well as the infusion levels, and as well by the specific products fitting with existing user habits. The computerized method builds upon existing user habits to prescribe and adjust timing of ingestion, frequency of ingestion, and quantity of ingestion of supplements without requiring the user to undertake new actions.

In another embodiment, the present computerized method and system can supplement consumer products, with supplementation determined based on user activities instead of health data. Where the above embodiments rely on medical-based health data, another embodiment can instead utilize data relating to the user activities. As noted above, the user activity can be acquired from any number of sources, including user tracking software/applications, consumer habit/consumer tracking data, user feedback such as survey response, social media tracking/feedback data, or any other suitable sources.

Therefore, the supplementation is not expressly limited to cure a medical or health-based deficiency.

In one embodiment, user activity data may indicate the user lives in a location where recreational usage and/or medicinal use of marijuana is legal, as well as the user activity indicating the user partakes in recreational or medical-based marijuana consumption. In this embodiment, the supplement may include THC or similar supplement. In another embodiment, the supplement may include CBD, providing for the medical benefits of THC without the psycho-tropic effect. Here, the supplementation can be infused into a consumer product offering a dosing or micro-dosing effect.

It is recognized the supplementation can be any suitable supplement capable of being infused into the consumer product. For example, for a person suffering from joint pain or other ailments, CBD oil or fish oils can be infused into the consumer product. In another example, a prescription or other controlled or regulated substance can be infused within a product. For example, a person may suffer from allergies, over-the-counter allergy medicine infused into a consumer product, e.g. shampoo or toothpaste. The user can then receive allergy medicine distributed via ingestion means consistent with existing habits.

The supplement or supplements used herein may also include non-prescription supplements. As used herein, non-prescription supplements can be any supplement or supplements not falling into a regulated, controlled, or prescribed category. Non-prescription supplements may also not be subject to recommended daily allowances and may also not be traceable in biological specimens acquired from the user. For example, non-prescription supplements can include, but are not limited to, one or more: ashwagandha, astragalus, chamomile, collagen, echinacea, elderberry, ginger, ginseng, L-theanine, melatonin, mint, N-acetyl-L-tyrosine, omega-3, sage, turmeric, valerian, by way of example.

Therefore, in this embodiment, supplementation and dosing levels are based on user activity data. In one embodiment, the user activity data can be acquired using one or more user interfacing functions. For example, the supplementation selection does not necessarily have to be an intuitive or data analysis function, but rather can be predicated or based on a user selection or user request. In this example, the user may fill out a form requesting supplementation, e.g. requesting one or more consumer products having allergy medicine infused therein, requesting one or more consumer products having CBD oil infused therein, etc.

FIGS. 1 through 4B are conceptual illustrations allowing for an explanation of the present invention. Notably, the figures and examples above are not meant to limit the scope of the present invention to a single embodiment, as other embodiments are possible by way of interchange of some or all of the described or illustrated elements. Moreover, where certain elements of the present invention can be partially or fully implemented using known components, only those portions of such known components that are necessary for an understanding of the present invention are described, and detailed descriptions of other portions of such known components are omitted so as not to obscure the invention. In the present specification, an embodiment showing a singular component should not necessarily be limited to other embodiments including a plurality of the same component, and vice-versa, unless explicitly stated otherwise herein. Further, the present invention encompasses present and future known equivalents to the known components referred to herein by way of illustration.

The foregoing description of the specific embodiments so fully reveals the general nature of the invention that others can, by applying knowledge within the skill of the relevant art(s) (including the contents of the documents cited and incorporated by reference herein), readily modify and/or adapt for various applications such specific embodiments, without undue experimentation, without departing from the general concept of the present invention. Such adaptations and modifications are therefore intended to be within the meaning and range of equivalents of the disclosed embodiments, based on the teaching and guidance presented herein.

1. A computerized method for electronically determining nutritional supplement delivery via consumer products, the method comprising:
receiving user data regarding a user, the user data including user health data and user activity data;
electronically processing the user health data relative to health marker data sets and determining a health deficiency of the user;
electronically processing the user activity data and determining a plurality of habits of the user;
based on the health deficiency of the user, electronically determining a supplement for addressing the health deficiency of the user;
electronically determining a plurality of consumer products, each of the plurality of consumer products having a consumption technique consistent with at least one of the plurality of habits of the user, each of the plurality of consumer products are capable of being supplemented with the supplement;
based on the user health data, calculating a dosing value for the supplement for addressing the health deficiency; and
for each of the consumer products, electronically determining a supplementation level for consumption of the consumer product consistent with the plurality of habits of the users such that consumption of the plurality of consumer products collectively meet the dosing value for the supplement.

2. The computerized method of claim 1 further comprising:
for at least one of the plurality of consumer products, transmitting the supplementation level to an infusion system; and
infusing the at least one consumer product with the supplement to generate the consumer product having the supplement contained therein consistent with the supplementation level.

3. The computerized method of claim 2, further comprising:
determining the supplementation level based on the at least one consumer product, the consumption technique associated therewith, and a supplement absorption rate based on the consumption technique.

4. The computerized method of claim 1 further comprising:
receiving a plurality of survey answers from the user in response to survey questions presented through a graphical user interface, wherein the plurality of survey answers include information relating to lifestyle data of the user; and
determining the health deficiency based on the lifestyle data.

5. The computerized method of claim 1, wherein the plurality of consumer products includes at least one of: toothpaste, mouthwash, shaving cream, hand lotion, body lotion, shampoo, and conditioner.

6. The computerized method of claim 1, wherein the supplement is a first supplement, the method further comprising:
determining a second supplement, wherein the second supplement is different from the first supplement; and
supplementing at least one of the consumer products with the second supplement, wherein the inclusion of the second supplement does not interfere with the first supplement.

7. The computerized method of claim 1
after a consumption duration, receiving, via the networked connection, second user data regarding the user, the second user data including second user health data; and
analyzing the second user health data relative to the first user health data to compute a variance value of the health deficiency after the consumption duration.

8. The computerized method of claim 7, wherein the consumption duration is at least one of: a time period for fully consuming the plurality of consumer products and a time period for medicinal effects of the supplement infused within the plurality of consumer product.

9. The computerized method of claim 1 further comprising:
after the consumption duration, submitting an electronic survey to the user, wherein the electronic survey includes at least one inquiry relating to the plurality of habits of the user; and
updating the user activity data based thereon.

10. The computerized method of claim 1 further comprising:
electronically generating delivery instructions for commercial delivery of each of the plurality of consumer products to the user;
wherein the electronically generating delivery instructions for commercial delivery further includes electronically transmitting product recommendation messages to the user via a commercial user interface and receiving an order request from the user via the commercial user interface for electronic purchase of at least one of the consumer products.

11. The computerized method of claim 1 further comprising:
based on the plurality of consumer products, electronically determining at least one supplementary consumer product associated therewith.

12. The computerized method of claim 1 further comprising:
generating a consumption protocol for the user based on the dosing value for the supplement and the plurality of consumer products, wherein the consumption protocol includes at least one of: timing instructions for consuming the consumer products and quantity instructions for consuming the consumer products.

13. A computerized method for electronically determining nutritional supplement delivery via consumer products, the method comprising:
receiving user data regarding a user, the user data including user activity data;
electronically processing the user activity data and determining at least one activity of the user and a plurality of habits of the user;
based on the at least one activity of user, determining a supplement for consumption by the user;
electronically determining a consumer product having a consumption technique consistent with at least one of the plurality of habits of the user, the consumer product is capable of being supplemented with the supplement;
for the consumer products, electronically determining a supplementation level for consumption of the consumer product consistent with the plurality of habits of the users such that consumption of the plurality of consumer products provides for a dosing level of the supplement; and
infusing the consumer product with the supplement.

14. The computerized method of claim 13, further comprising:
determining the supplementation level based on the at least one consumer product, the consumption technique associated therewith, and a supplement absorption rate based on the consumption technique.

15. The computerized method of claim 13 further comprising:
receiving a plurality of survey answers from the user in response to survey questions presented through a graphical user interface, wherein the plurality of survey answers include information relating to lifestyle data of the user; and
processing the user activity data in combination with the lifestyle data.

16. The computerized method of claim 13, wherein the plurality of consumer products includes at least one of: toothpaste, mouthwash, shaving cream, hand lotion, body lotion, shampoo, and conditioner.

17. The computerized method of claim 13, wherein the supplement is a controlled substance.

18. The computerized method of claim 13, wherein the supplement is based off a medical prescription.

19. The computerized method of claim 13, wherein the supplement is a non-prescription supplement.

20. The computerized method of claim 19, wherein the non-prescription supplement includes at least one of: ashwagandha, astragalus, chamomile, collagen, echinacea, elderberry, ginger, ginseng, L-theanine, melatonin, mint, N-acetyl-L-tyrosine, omega-3, sage, turmeric, and valerian.
